(19) United States
(12) Patent Application Publication
(10) Pub. No.: US 2021/0047860 A1
(43) Pub. Date: Feb. 18, 2021

(54) DOOR HANDLE ASSEMBLY

(71) Applicant: Brian Grau, St. Louis, MT (US)

(72) Inventor: Brian Grau, St. Louis, MT (US)

(21) Appl. No.: 17/087,010

(22) Filed: Nov. 2, 2020

Related U.S. Application Data
(60) Provisional application No. 63/047,015, filed on Jul. 1, 2020.

(51) Int. Cl. E05B 1/00; A61L 2/26; A61L 2/18; A01N 25/34

(52) U.S. Cl. CPC E05B 1/0069; E05B 1/0015; E05B 1/0061; A61L 2202/14; A61L 2/18; A01N 25/34; A61L 2/26

(57) ABSTRACT

The present invention is directed to a door handle assembly that reduces the cross-transmission of pathogens. The door handle assembly includes a panel having a front face and a rear face; a bracket is configured on the front face; a cartridge is interchangeably received by the bracket, wherein the cartridge includes a plurality of sheets, the cartridge configured to dispense a single sheet from the plurality of sheets through a longitudinal slot that is configured in the cartridge; and a handle coupled to the front face of the panel adjacent the bracket, wherein the cartridge and the handle are positioned such as the sheet is dispensed over the handle.

### CROSS-REFERENCE TO RELATED APPLICATIONS

This application claims priority to the U.S. provisional patent application Ser. No. 63/047,015, filed on Jul. 1, 2020, which is incorporated herein by reference in its entirety.

### FIELD OF INVENTION

The present invention generally relates to a door handle assembly, more particularly, the present invention relates to a door handle assembly that reduces transmission of microbes amongst users contacting a handle.

### BACKGROUND

Microbes that cause infection in human beings are called pathogens. Humans can contract infections from various sources including an infected person. Many pathogens can transmit from one person to another. For example, the influenza virus can quickly infect a number of persons. Hands contaminated with secretions/excretions from an infected person are a major source spread of infection. For example, secretions from the eyes, nose, and mouth can easily transmit to a hand while the infected person sneezes, coughs, or rubs the eyes. Pathogens can survive on the hands for variable durations depending upon the type of pathogen. The pathogens can transmit to clean surfaces touched by the contaminated hands. During the duration in which pathogens are alive on a surface, clean hands can pick the pathogens from the contaminated surfaces.

Therefore, hand hygiene is essential for wellbeing and can prevent the spread of many infections. The Covid-19 outbreak emphasized the need of individual hygiene for overall wellbeing and safety. Washing hands thoroughly with soap and water can help in maintaining the hand hygiene but washing the hands frequently can be impractical.

Some articles frequently come in contact with many people. A handle is one such object that is frequently handled by several people. For example, the refrigerator handle can be touched by almost all family members. Similarly, door handles come in contact with a number of people. Contaminated handle surfaces can easily transmit pathogens to a number of people.

Therefore, a need is appreciated for an assembly that reduces the transmission of pathogens to a handle surface. More preferably, a need is appreciated for an assembly the reduces cross-transmission of pathogens from contaminated handles.

### SUMMARY OF THE INVENTION

The principal object of the present invention is therefore directed to a door handle assembly that reduces cross-transmission of the pathogens.

It is a further object of the present invention that the door handle assembly can prevent the transmission of pathogens to a handle's surface.

It is an additional object of the present invention that door handle assembly reduces the chances of cross-transmission of the infection.

It is another object of the present invention that the door handle assembly is easy to install.

It is a further object of the present invention that the door handle assembly is economical to manufacture.

It is yet another object of the present invention that the door handle assembly can retrofit to a conventional door.

In one aspect, disclosed is a door handle assembly that reduces the cross-transmission of pathogens. The door handle assembly includes a panel having a front face and a rear face; a bracket is configured on the front face; a cartridge can be interchangeably received by the bracket, wherein the cartridge includes a plurality of sheets, the cartridge configured to dispense a single sheet from the plurality of sheets through a longitudinal slot configured in the cartridge; and a handle coupled to the front face of the panel below the bracket, wherein the cartridge and the handle are positioned such as the sheet is dispensed over the handle.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a perspective view of the door holder assembly, according to an exemplary embodiment of the present invention.

FIG. 2 shows a sheet dispensed from the cartridge over the handle, according to an exemplary embodiment of the present invention.

FIG. 3 is an exploded view of the door handle assembly, also showing the sheet folded over the handle, according to an exemplary embodiment of the present invention.

FIG. 4 shows the handle grabbed by a hand, according to an exemplary embodiment of the present invention.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as devices and methods of use thereof. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

The following detailed description is described with reference to the drawings, wherein like reference numerals are used to refer to like elements throughout. In the following description, for purposes of explanation, specific details may be set forth in order to provide a thorough understanding of the subject innovation. It may be evident, however, that the claimed subject matter may be practiced without these specific details.

Disclosed is a door handle assembly for reducing cross-contamination of pathogens among people using a handle. The disclosed door handle assembly also prevents contamination of the handle from an infected person. The assembly disclosed herein is particularly advantageous during an epidemic, such as the Covid-19 epidemic which affected millions of people across the globe. Moreover, the door handle assembly disclosed herein helps to keep the hand hygiene.

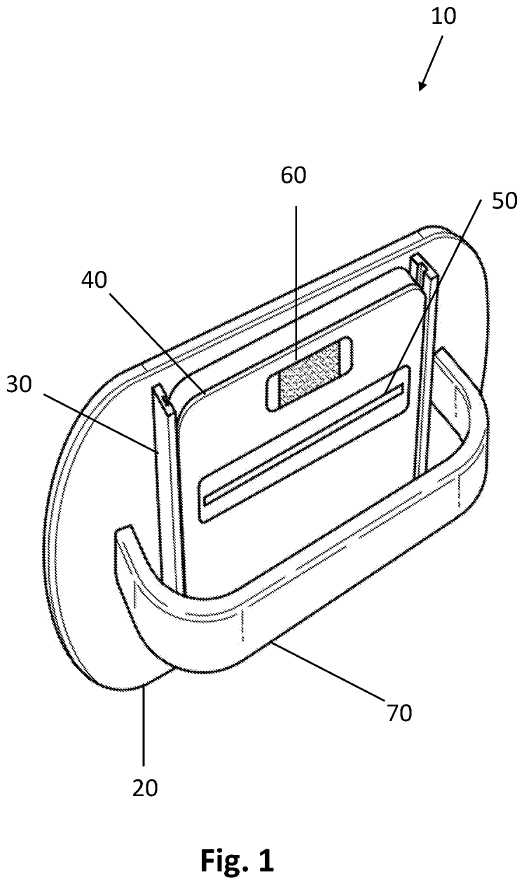

Now referring to FIG. 1 which shows a preferred embodiment of the disclosed door handle assembly 10. The assembly 10 includes a panel 20 which is shown to be of an elongated planar configuration with round edges. The panel 20 is having a front face and a rear face. The rear face contacts the door surface when the assembly 10 is mounted on a door. The panel 20 can be made of any rigid and durable material, such as metal. For example, the panel 20 can be provided with holes at the corners through which fasteners can be inserted to secure the assembly to the door. Furthermore, shown in FIG. 1 is a bracket 30 configured on the front face of the panel 20. The bracket 30 is configured to interchangeably receive a cartridge 40. The cartridge is having a longitudinal slot 50 and a pull strip 60. A handle 70 can also be seen coupled to the front face of the panel 20 below the bracket.

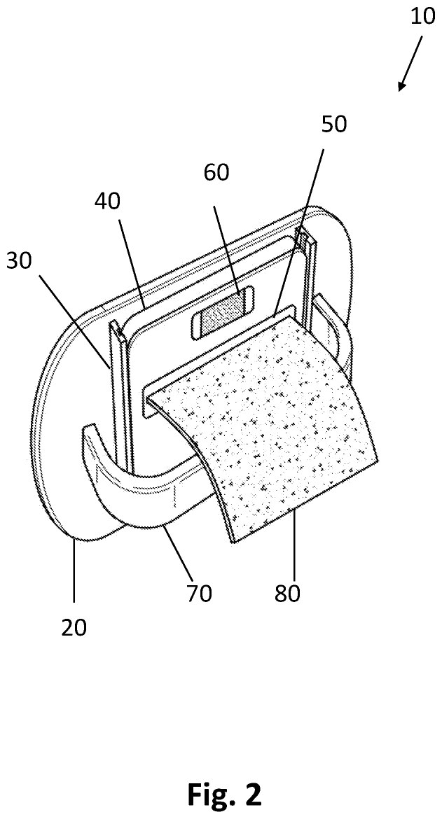

The cartridge can be a dispenser that can dispense a sheet. FIG. 2 shows a sheet 80 extended out from slot 50 of the cartridge and positioned over the handle 70. The sheet can be similar to a tissue paper or any other sheet made of paper, fabric, and like material. The sheets can also be embedded with disinfectant, provided that the disinfectant is compatible with human skin. The dimension of the sheet is such as a single sheet can fold over the handle. In one case, the width of the sheet is proportional to the length of the handle. The cartridge includes a number of sheets that can be dispensed one at a time through the slot in the cartridge. In one case, the number of sheets can be a roll of sheet, wherein the cartridge includes a means of cutting the sheet of the desired length. In another case, the number of sheets can be a web of sheets that can be interconnected by a line of weakness, such as spaced perforations. Each sheet is having a leading end and a trailing end. The leading end of the sheet extends out of the dispenser while the trailing end is loosely held at the slot. This prevents the sheet from dropping, wherein the grabbing of the handle causes the sheet to fold over the handle. Folding of the sheet over the handle releases the sheet from the slot.

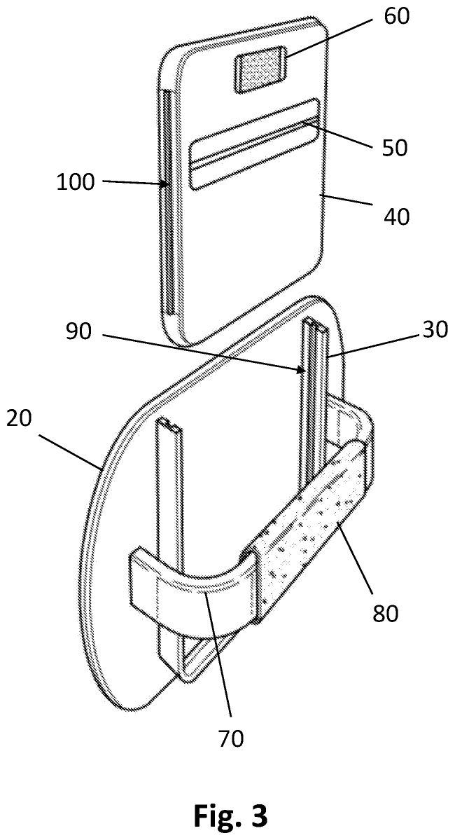

The cartridge can be interchangeably inserted into the bracket. FIG. 3 shows an exploded view of the door handle assembly. The bracket 30 is shown to have parallel and opposite longitudinal slots 90. The cartridge is also having corresponding protrusions 100 that can slidably engage with the slots 90. This allows the cartridge to slidably and removably insert into the bracket. The cartridge can have dispensing means to dispense a single sheet. For example, the cartridge can have a sensor that detects an approaching person and upon detecting, automatically dispense a single sheet over the handle. Alternatively, pulling out one sheet causes the leading end of the adjacent sheet to extend out from the slot.

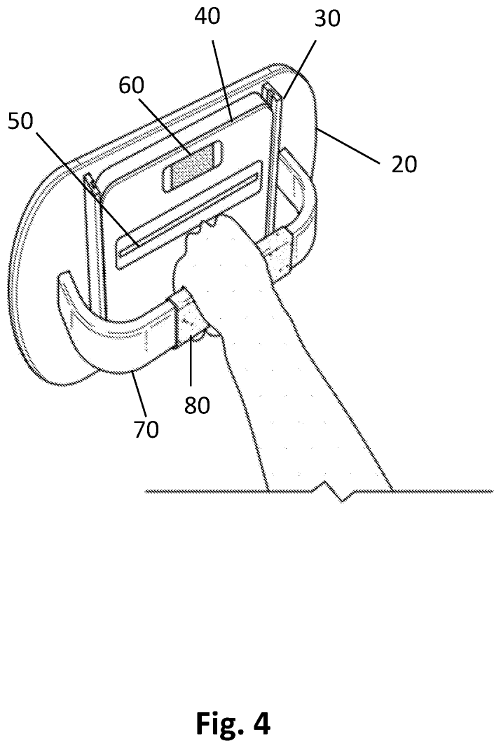

FIG. 4 shows the door handle assembly 10 having the panel 20, the bracket 30 configured on the front face of the panel 20. The cartridge 40 inserted into the bracket 30, wherein the cartridge is having the slot 50 for dispensing a tissue paper, and a pull strip 60. A handle 70 located below the slot, such as the tissue paper can be dispensed over the handle. A hand can be seen grabbed to the handle wherein the tissue is folded over the handle. The tissue paper prevents any contact of the hand with the handle. The width of the tissue paper is proportional to the length of the handle, such as the tissue paper can cover the handle so that the hand does not contact the handle's surface. Upon removing the hand from the handle, the folded tissue also loosens up and could be picked by the hand. The tissue can then be disposed of, for example, thrown in a dust bin.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A door handle assembly comprising:
a panel having a front face and a rear face;
a cartridge having a plurality of sheets, the cartridge configured to dispense a sheet from the plurality of sheets through a slot, the slot is configured in the cartridge;
a bracket configured on the front face of the panel, the bracket configured to interchangeably receive the cartridge; and
a handle configured on the front face of the panel and below the bracket, wherein the position of the handle and the slot is such that the sheet is dispensed from the cartridge over the handle.

2. The door handle assembly of claim 1, wherein the sheet is a tissue paper embedded with disinfectant.

3. The door handle assembly of claim 1, wherein the bracket comprises a pair of slots and the cartridge comprises protrusions configured to removably and slidably couple with the pair of slots.

4. The door handle assembly of claim 1, wherein the width of the plurality of sheets is proportional to the length of the handle.

5. The door handle assembly of claim 1, wherein the cartridge further comprises a sensor to detect a person in proximity of the door handle assembly, wherein the cartridge is configured to dispense the sheet upon detecting the presence of the person.
